SEQUENCE LISTING (6 sequences)

SEQ ID NO: 1 - DNA, 1812 bp, Rhodococcus ruber
  source 1..1812: mol_type = genomic DNA; strain = SC1
atgacgacgagcatcgaccgtgaagcgctccggaggaagtatgcggaagagcgcgacaagcggatccgaccggacggaaacgaccagtacattcgcctcgatcacgtggacggctggtcgcatgacccgtacatgccgatcacgccccgcgaacccaagctcgaccatgtgacgttcgcattcatcggcggcggcttctccggcctggtcaccgccgcacgccttcgagaatccggagtcgagagcgtccgcatcatcgacaaggccggcgacttcggtggcgtctggtactggaacagataccccggcgcgatgtgtgacaccgcagccatggtgtacatgccgctgctcgaggaaaccggctacatgcccaccgagaagtacgcgcacggcccggagatcctcgaacactgccaacgaatcggcaaacactacgacttgtacgacgacgcgctgttccacaccgaagtcaccgacctggtctggcaggagcacgatcagcgctggcggatctcgacgaaccgaggtgaccacttcacggcccaattcgtgggtatgggcaccggccctctgcacgtggcgcagctgccgggcatccccgggatcgaatcgttccgcggcaagtcgttccacaccagtcgatgggactacgactacaccggcggcgacgcgctgggcgcgccgatggacaagctcgcggacaagcgcgtagcggtgatcggaaccggcgcgaccgcggtgcagtgcgtgcccgaactggccaagtactgcagggaactgtacgtcgtccaacgcacgccgtcggcggtcgacgaacggggaaaccacccgatcgacgagaagtggttcgcgcagatcgcgacacccggttggcagaagcgctggctggacagtttcaccgccatctgggacggcgtgctcaccgacccgagcgagttggcgatcgaacacgaggacctcgtccaggacgggtggaccgcgctcggccagaggatgcgtgcagccgtcggatccgtgccgatcgagcagtactcgccggaaaatgtgcagcgggcactcgaggaggccgacgacgagcagatggagcgcatccgcgcccgcgtcgacgagatcgtcaccgatcccgccactgccgcacagctcaaggcctggttccgtcagatgtgcaagcgaccgtgcttccatgacgactacctgccggcgttcaatcggcccaacacacatctcgtcgacacgggcggcaaaggggtggagcgcatcaccgagaacggcgtggtcgttgccggggtggagtacgaggtggactgcatcgtctacgcctccgggttcgaattcctcggcaccggctacaccgaccgtgccggattcgacccgacgggacgcgacggggtcaagctgtcggagcattgggcgcagggcacacgaaccctccacggcatgcacacctacggattccccaacctgttcgtgctccagttgatgcagggcgcagctctcggatcgaacattccccacaacttcgtcgaagccgctcgcgtcgtcgctgcgatagtcgatcacgtgctgagcaccggcacatccagcgtcgagacgacgaaggaggccgagcaggcgtgggtgcagcttctcctcgaccacggccggcccctcggtaatcccgaatgcactccgggctactacaacaacgaaggcaaacccgccgaactgaaggatcggctcaacgtcggctatccggcaggctcggcagcgttcttccgcatgatggaccactggcttgcggccggcagcttcgacggcctcaccttccgctga

SEQ ID NO: 2 - DNA, 1812 bp, synthetic construct
  misc_feature 1..1812: note = Artificial sequence
  source 1..1812: mol_type = other DNA
atgacgaccagcattgaccgtgaagccctgcgccgtaagtatgccgaagaacgtgataaacgcatccgtccggatggcaacgatcagtacatccgtctggaccatgtcgatggctggagccatgacccgtacatgcctattaccccacgcgagccgaaactggaccacgtcacgttcgcgttcattggtggcggtttttcgggtctggtcaccgctgcccgtctgcgcgagagcggcgttgagagcgtgcgtatcatcgacaaggcgggtgattttggcggcgtgtggtactggaaccgttatccgggtgcgatgtgcgataccgcggcaatggtgtatatgccgttgctggaagaaacgggttatatgccgaccgagaagtacgcccacggtccggaaattctggaacactgtcaacgtatcggtaaacattacgatctgtatgatgacgctctgttccataccgaggtcaccgacctggtgtggcaagaacacgaccagcgttggcgcatcagcacgaatcgtggtgatcatttcacggcgcagtttgttggtatgggtacgggcccactgcacgtggcgcaactgccgggcattccgggcattgagtctttccgtggtaaatcctttcatacctctcgctgggactacgattataccggtggcgatgcactgggtgcaccgatggacaagctggcagacaagcgcgttgcggtcatcggtactggcgccaccgcggtccagtgtgttccggagctggcgaaatactgccgtgagctgtatgtcgttcagcgcaccccgagcgcggttgacgaacgcggcaaccacccgattgacgagaaatggttcgcgcagattgcaacgccgggttggcaaaagcgttggctggacagctttaccgcgatttgggatggtgttctgaccgacccgagcgagctggcaattgaacacgaggatctggtccaagacggctggacggcactgggccagcgcatgcgtgcagcggtgggtagcgtgccgatcgaacaatacagcccggagaatgttcagcgtgcgttggaagaggcagatgacgaacaaatggagcgcatccgtgcgcgcgtggatgagatcgtgacggacccggctaccgcggctcaactgaaggcgtggttccgccaaatgtgcaaacgtccttgctttcacgatgactatttgcctgcgttcaatcgtccgaatacccacctggttgatacgggcggtaagggcgttgaacgtatcaccgagaatggtgtggttgttgcgggtgtggaatacgaagtggattgcattgtttacgcgagcggttttgaatttctgggtaccggttacacggaccgtgccggcttcgacccgacgggtcgcgacggtgtgaaactgtctgagcactgggcgcagggtacccgtaccctgcacggtatgcacacctacggttttccaaacttgttcgttctgcagctgatgcagggtgcggccttgggctccaacatcccgcacaacttcgttgaggcagcgcgtgtcgtggccgccattgtagatcacgtcctgagcacgggcactagcagcgtggagactaccaaagaggcggaacaggcatgggtccaattgttgctggatcatggtcgtccgctgggcaatccggagtgtaccccgggttattacaacaacgagggtaagccggcagagctgaaagatcgtctgaatgtcggctatccggctggttccgctgctttctttcgcatgatggaccattggttggccgcaggcagctttgacggcctgactttccgttaa

SEQ ID NO: 3 - protein, 603 aa, Rhodococcus ruber
  source 1..603: note = Strain SC1
MTTSIDREALRRKYAEERDKRIRPDGNDQYIRLDHVDGWSHDPYMPITPREPKLDHVTFAFIGGGFSGLVTAARLRESGVESVRIIDKAGDFGGVWYWNRYPGAMCDTAAMVYMPLLEETGYMPTEKYAHGPEILEHCQRIGKHYDLYDDALFHTEVTDLVWQEHDQRWRISTNRGDHFTAQFVGMGTGPLHVAQLPGIPGIESFRGKSFHTSRWDYDYTGGDALGAPMDKLADKRVAVIGTGATAVQCVPELAKYCRELYVVQRTPSAVDERGNHPIDEKWFAQIATPGWQKRWLDSFTAIWDGVLTDPSELAIEHEDLVQDGWTALGQRMRAAVGSVPIEQYSPENVQRALEEADDEQMERIRARVDEIVTDPATAAQLKAWFRQMCKRPCFHDDYLPAFNRPNTHLVDTGGKGVERITENGVVVAGVEYEVDCIVYASGFEFLGTGYTDRAGFDPTGRDGVKLSEHWAQGTRTLHGMHTYGFPNLFVLQLMQGAALGSNIPHNFVEAARVVAAIVDHVLSTGTSSVETTKEAEQAWVQLLLDHGRPLGNPECTPGYYNNEGKPAELKDRLNVGYPAGSAAFFRMMDHWLAAGSFDGLTFR

SEQ ID NO: 4 - DNA, 1806 bp, Pseudomonas sp.
  source 1..1806: mol_type = genomic DNA; strain = HI-70
atgagtcagctaattcaagagccggccgaggctggggtaacgtcgcagaaagtttccttcgatcatgtggcgcttcgcgaaaaatatcgtcaggagcgcgacaagcgcttgcgtcaggacggccaagagcaatatctggaagtcgccgtcacatgtgacgaatacctgaaagacccctatgccgatccgatcgtgcgtgatccggtggtgcgcgagaccgacgtgttcatcatcggtggcggtttcggtggtctgttggctgcggtacgcctgcagcaagcaggcgttagcgattatgtgatggttgagcgcgccggtgactatggcggcacctggtactggaaccgctacccgggggcgcagtgcgacatcgagtcgtatgtctacatgcccttgctcgaagaaatgggttacataccgaccgagaagtacgccttcggtacggagatcctcgagtactccagatcaatcggccgcaaatttggcttgtacgagcgcacctacttccagactgaagtgaaggatctgagctgggacgatgaagcggcccgctggcgcattaccaccgaccgcggcgacaagttcagcgcgcgcttcgtgtgcatgtccaccggccccttgcagcggcccaaactgcccggcatcccaggtatcacgtccttcaagggccactccttccacaccagtcgctgggactactcctataccggcggcgatcagaccggcaacctggaaggtttgaaagacaagcgcgtggccatcatcggtaccggcgccacctcaatccaggctgtgccacacctggcggcctatgcccaagagctgtatgtcatccagcgcacgccaatttccgtgggcttccgtggcaacaagccgactgatcctgaatgggccaagagcctgcagccgggttggcagcaagcgcgtatggacaacttcaatgcgatcacccacggcatgccggtcgatgtcgatctggtccaggacagctggaccaagatcttcggcgaaatcggcgtttttctgggttccgatggcagccgtgcgcagatggtcgacttccagttgatggagcaaatccgcgcccgcgtcgatcaggaagtcaaggatccggccaccgctgagtcgctcaagccttactacaacatcatgtgtaagcgtccgggcttccatgacagttatctgccctccttcaacaagcccaatgtcaccctggtcgatacccaaggcgctggtgttgagcgcatcaccgaaaagggcctggtggtcaacggccgcgaatatgaagtcgactgcctgatctacgccaccggcttcgagtaccagaccaagttgtcgcgccgcaatggctacgaaatccacgggcgcaatggccagccgctgagtgacaagtggaaagacggcctgtccacactgtggggctaccacattcgtgacttcccgaactgcttcatccttggcaatggtcagtctgcggtaacaccgaacttcactcacatgctcaacgaagctggcaagcatgtggcctatgtggtcaagcactgcctggacgagcgcgtcgatgtcttcgagccgaccgcagaggctgagcaggcgtgggttgaccacgtcatgtcgttcgctgggatcaagcagcaatacgaccgcgagtgcaccccgagttactacaacaacgaaggccaggtgaacgacgttgcgctgacccgcaacaacttctacccgggcggtgcggtcgccttcataaacattctgcgggagtggcgagagaagggcgatttcgcgcagttccagcagcgcaagcgttga

SEQ ID NO: 5 - DNA, 1806 bp, synthetic construct
  misc_feature 1..1806: note = Artificial sequence
  source 1..1806: mol_type = other DNA
atgagccaactgatccaagaaccagcagaagcaggtgtgacgtcccagaaagtttcgtttgaccatgtagccctgcgcgagaagtaccgccaagagcgtgacaagcgtctgcgccaagatggccaggagcagtacctggaagtcgcggttacctgtgacgagtacctgaaagacccgtatgccgatccgatcgtccgcgatccggtggttcgtgaaacggatgttttcattattggtggtggttttggtggtttgctggccgcagttcgcctgcaacaagcgggcgtgagcgactatgtcatggttgagcgtgcgggtgattacggtggtacctggtattggaatcgttaccctggtgcacagtgcgacatcgagagctatgtttacatgccattgctggaagagatgggttacatcccgaccgaaaagtatgcctttggcacggaaatcttggagtattcgcgttccattggccgtaaattcggtctgtacgaacgtacgtactttcaaaccgaggtcaaagacctgagctgggacgacgaggctgcacgttggcgcattacgaccgatcgtggcgacaaattcagcgcgcgttttgtgtgtatgagcactggtccgttgcagcgcccgaaattgcctggtattccgggcattaccagcttcaagggtcattcttttcacactagccgttgggactactcttataccggcggtgatcagaccggtaatctggaaggtctgaaagataagcgtgttgcgatcatcggtaccggtgcgacgagcattcaggctgttccgcacctggcggcatacgcgcaagagctgtatgttattcaacgtaccccgatcagcgttggctttcgcggtaataagccgaccgacccggagtgggcgaagtccctgcagccgggttggcagcaagcacgcatggacaatttcaacgcaatcacgcacggtatgccggtcgacgtggatctggttcaggatagctggacgaaaatcttcggcgagatcggcgttttcctgggcagcgatggctcccgtgcgcaaatggttgatttccagctgatggagcaaatccgcgctcgtgtcgatcaagaagtcaaagatccggccaccgcggaaagcctgaagccgtactataacatcatgtgcaagcgtccgggtttccatgacagctatctgccgagcttcaacaagccgaatgtcactctggttgacacccagggtgcgggtgtggagcgcattaccgagaaaggtctggtcgtgaacggtcgcgagtacgaggtggattgcctgatttatgccaccggcttcgagtaccagacgaagctgtctcgtcgcaacggctacgagatccacggccgtaatggtcagccattgagcgacaagtggaaagatggcctgagcaccctgtggggttatcacattcgtgactttccgaactgcttcattctgggcaatggtcaaagcgcagtcaccccgaacttcacccacatgctgaatgaagcgggcaaacatgtggcttacgtggtgaaacactgcttggatgaacgcgtcgatgtctttgaaccgacggcagaggctgagcaggcgtgggtggaccatgtgatgagctttgcgggcattaagcagcagtacgaccgcgaatgtacgccgagctattacaataacgaaggccaagtgaacgacgtggcgctgacccgtaataacttttacccgggtggcgccgtagcgttcattaacatcctgcgtgagtggcgtgaaaaaggtgattttgcgcaattccagcagcgtaagcgttaa

SEQ ID NO: 6 - protein, 601 aa, Pseudomonas sp.
  source 1..601: note = Strain HI-70
MSQLIQEPAEAGVTSQKVSFDHVALREKYRQERDKRLRQDGQEQYLEVAVTCDEYLKDPYADPIVRDPVVRETDVFIIGGGFGGLLAAVRLQQAGVSDYVMVERAGDYGGTWYWNRYPGAQCDIESYVYMPLLEEMGYIPTEKYAFGTEILEYSRSIGRKFGLYERTYFQTEVKDLSWDDEAARWRITTDRGDKFSARFVCMSTGPLQRPKLPGIPGITSFKGHSFHTSRWDYSYTGGDQTGNLEGLKDKRVAIIGTGATSIQAVPHLAAYAQELYVIQRTPISVGFRGNKPTDPEWAKSLQPGWQQARMDNFNAITHGMPVDVDLVQDSWTKIFGEIGVFLGSDGSRAQMVDFQLMEQIRARVDQEVKDPATAESLKPYYNIMCKRPGFHDSYLPSFNKPNVTLVDTQGAGVERITEKGLVVNGREYEVDCLIYATGFEYQTKLSRRNGYEIHGRNGQPLSDKWKDGLSTLWGYHIRDFPNCFILGNGQSAVTPNFTHMLNEAGKHVAYVVKHCLDERVDVFEPTAEAEQAWVDHVMSFAGIKQQYDRECTPSYYNNEGQVNDVALTRNNFYPGGAVAFINILREWREKGDFAQFQQRKR

(19) United States
(12) Patent Application Publication
(10) Pub. No.: US 2026/0028654 A1
(43) Pub. Date: Jan. 29, 2026

(54) BIOTECHNOLOGICAL PRODUCTION OF MACROCYCLIC LACTONES

(71) Applicant: SYMRISE AG, Holzminden (DE)

(72) Inventors: Stefan LAMBRECHT, Hehlen (DE); Johannes PANTEN, Höxter (DE); Dirk FABRITIUS, Ansbach (DE); Martin SCHÜRMANN, Jülich (DE); Henricus Martinus Maria Gerardus STRAATMANN, HORST (NL)

(21) Appl. No.: 18/998,108

(22) PCT Filed: Jul. 26, 2022
(86) PCT No.: PCT/EP2022/070861

§ 371 Date: Jan. 23, 2025

(51) Int. Cl. C12P 17/08; C07D 313/00

(52) U.S. Cl. CPC C12P 17/08; C07D 313/00

(57) ABSTRACT

The present invention relates to methods for converting unsaturated macrocyclic ketones to lactones wherein the conversion is catalyzed by a Baeyer-Villiger monooxygenase (BVMO). The invention also relates to the use of a Baeyer-Villiger monooxygenase for the conversion of unsaturated macrocyclic ketones to lactones and provides certain novel macrocyclic lactones, which are accessible by the method according to the invention. Furthermore, the present invention relates to the use of novel and known macrocyclic lactones as fragrances.

The present invention relates to methods for converting unsaturated macrocyclic ketones to lactones wherein the conversion is catalyzed by a Baeyer-Villiger monooxygenase (BVMO). The invention also relates to the use of a Baeyer-Villiger monooxygenase for the conversion of unsaturated macrocyclic ketones to lactones and provides certain novel macrocyclic lactones, which are accessible by the method according to the invention. Furthermore, the present invention relates to the use of novel and known macrocyclic lactones as fragrances.

Macrocyclic “musk” lactones represent interesting compounds for application in the fragrance industry. Some macrocyclic lactones are already known to have an attractive fragrance but further variation is highly desirable in order to discover novel compounds and assess their potential for the fragrance industry. In particular, a double bond in various positions of the macrocycle can provide interesting changes to the olfactory properties of the molecules and thus provide novel olfactory aspects.

Synthetic Methods have been described to obtain unsaturated lactones by metathesis, but this procedure is rather complicated. As a precursor to macrocyclic lactones, the corresponding macrocyclic ketones are readily available. However, chemical oxidation of unsaturated ketones is highly unselective and leads to the epoxidation of the double bond as a frequent side reaction. Trials have been done to oxidize unsaturated cyclic ketones in zeolites, and it was possible to reach a much better selectivity for oxidation of the carbonyl group this way. However, this method only worked for small molecules since presumably, macrocyclic compounds are too large to fit into the pores of the zeolite.

It is known from the literature that macrocyclic ketones can be oxidized to lactones by a Baeyer-Villiger monooxygenase (Kostichka et al., Journal of Bacteriology, 2001, Vol. 183, No. 21, 6478-6486; Iwaki et al., Applied and Environmental Microbiology, 2006, Vol. 72, No. 4, 2707-2720, Schumacher, Fakoussa, Appl. Microbiol. Biotechnol., 1999, 52, 85-90). It has also been described that unsaturated cyclic systems can be converted under biotechnological conditions to lactones (Reignier et al., Chem. Commun., 2014, 50, 7793-7796). However, this reaction was only performed with rather small unsaturated rings and it was mentioned that side reactions (oxidation of the double bond) are possible.

There is therefore still a need to access unsaturated macrocyclic lactones by enzymatic conversion to be able to provide various (novel) fragrance molecules.

It was an objective of the present invention to provide methods to convert unsaturated macrocyclic ketones to the corresponding lactones while avoiding oxidation of the double bond.

The methods should be highly selective, applicable to a number of different macrocyclic systems and provide reasonable yields.

The above objectives are met by a method for converting one or a mixture of two or more compound(s) of formula I to one or a mixture of two or more compound(s) of formula II and/or III:

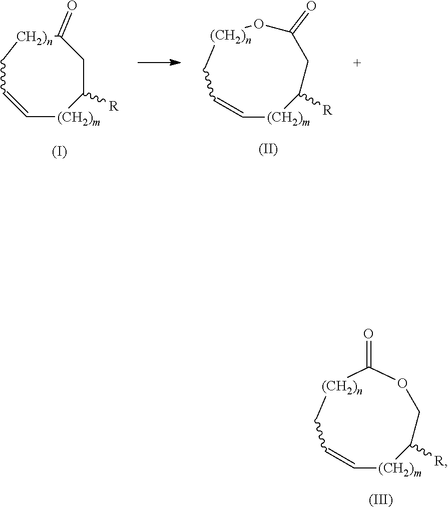

wherein the compound(s) of formula (I) is/are selected from the group consisting of the compounds (1) to (9):

| compound | n | m | R |
|---|---|---|---|
| (1) | 6 | 4 | H |
| (2) | 9 | 1 | H |
| (3) | 7 | 3 | H |
| (4) | 8 | 2 | H |
| (5) | 10 | 0 | H |
| (6) | 9 | 0 | H |
| (7) | 9 | 0 | CH₃ |
| (8) | 8 | 1 | CH₃ |
| (9) | 7 | 1 | CH₃ |

wherein the conversion is catalyzed by a Baeyer-Villiger monooxygenase, with the proviso that, in case only compound (1) is converted, the conversion is performed with whole cells expressing the Baeyer-Villiger monooxygenase.

Surprisingly, it was found out that when using Baeyer-Villiger monooxygenases (BVMO), it was possible to convert the above defined unsaturated ketones to lactones with a high selectivity for the oxidation of the carbonyl function and with good yields. The reaction may be performed either with cell-free enzyme formulations or with whole cells expressing the Baeyer-Villiger monooxygenase.

The compound (1) is also known as Globanone® and the compound (2) as Velvione®. Compound (3) to (5) represent isomers of a compound also known as Aurelione®, i.e. Aurelione 1, Aurelione 2 and Aurelione 3. Compound (6) is also known as Exaltenone and the compounds (7) and (8) as Muscenone®, d.h. Muscenone 1 and Muscenone 2. Compound (9) is also known as Cosmone®. All of the compounds (1) to (9) can be in E or Z configuration at the double bond.

A “Baeyer-Villiger monooxygenase” or “BVMO” in the context of the present disclosure is an enzyme belonging to the class of oxidoreductases. It catalyzes the oxidation of linear, cyclic and aromatic ketones to the corresponding esters or lactones.

In a preferred embodiment of the method described above, a mixture of the compounds (1) and (3), optionally further comprising compound(s) (4) and/or (5), is converted.

In a further preferred embodiment of the method according to any of the embodiments described above, the Baeyer-Villiger monooxygenase is a cyclopentadecanone monooxygenase (CPDMO) derived from a Pseudomonas species or a cyclododecanone monooxygenase (CDMO) derived from a Rhodococcus species.

Suitable Baeyer-Villiger monooxygenases were described by Kostichka et al. (Journal of Bacteriology, 2001, Vol. 183, No. 21, 6478-6486), Iwaki et al. (Applied and Environmental Microbiology, 2006, Vol. 72, No. 4, 2707-2720) and Schumacher and Fakoussa (Appl. Microbiol. Biotechnol., 1999, 52, 85-90). The genomic DNA sequences encoding the amino acid sequences of the enzymes derived from Rhodococcus ruber SC1 and Pseudomonas sp. HI-70 may be optimized for expression in a host cell.

In a preferred embodiment, the Baeyer-Villiger monooxygenase has an amino acid sequence selected from the sequences of SEQ ID NOs: 3 and 6, or an amino acid sequence having a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% to any of the sequences of SEQ ID NOs: 3 and 6.

The sequences of SEQ ID NO: 3 represents the amino acid sequence of the CDMO enzyme of Rhodococcus ruber SC1 and the sequence of SEQ ID NO: 6 represents the amino acid sequence of the CPDMO enzyme of Pseudomonas sp. HI-70.

In a further preferred embodiment of the method described in any of the embodiments above, the Baeyer-Villiger monooxygenase is encoded by a nucleic acid sequence selected from the sequences of SEQ ID NOs: 1, 2, 4 and 5, or a nucleic acid sequence having a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% to any of the sequences of SEQ ID NOs: 1, 2, 4 and 5.

The sequence of SEQ ID NO: 1 represents the CDMO encoding sequence in the genome of Rhodococcus ruber SC1, while the sequence of SEQ ID NO: 2 represents the same sequence optimized for expression in Escherichia coli both coding for the same amino acid sequence of SEQ ID NO: 3. The sequence of SEQ ID NO: 4 represents the CPDMO encoding sequence in the genome of Pseudomonas sp. HI-70, while the sequence of SEQ ID NO: 5 represents the same sequence optimized for expression in Escherichia coli both coding for the same amino acid sequence of SDEQ ID NO: 6.

Whenever the present disclosure relates to the percentage of identity of nucleic acid or amino acid sequences to each other these values define those values as obtained by using the EMBOSS Water Pairwise Sequence Alignments (nucleotide) programme (www.ebi.ac.uk/Tools/psa/emboss_water/nucleotide.html) nucleic acids or the EMBOSS Water Pairwise Sequence Alignments (protein) programme (www.ebi.ac.uk/Tools/psa/emboss_water/) for amino acid sequences. Alignments or sequence comparisons as used herein refer to an alignment over the whole length of two sequences compared to each other. Those tools provided by the European Molecular Biology Laboratory (EMBL) European Bioinformatics Institute (EBI) for local sequence alignments use a modified SmithWaterman algorithm (see www.ebi.ac.uk/Tools/psa/and Smith, T. F. & Waterman, M. S. “Identification of common molecular subsequences” Journal of Molecular Biology, 1981 147 (1):195-197). When conducting an alignment, the default parameters defined by the EMBL-EBI are used. Those parameters are (i) for amino acid sequences: Matrix=BLOSUM62, gap open penalty=10 and gap extend penalty=0.5 or (ii) for nucleic acid sequences: Matrix=DNAfull, gap open penalty=10 and gap extend penalty=0.5.

In one embodiment of the method described in any of the embodiments above, the conversion is performed with a cell-free enzyme formulation comprising the Baeyer-Villiger monooxygenase.

A cell-free enzyme formulation or cell-free extract comprises the enzyme, i.e. Baeyer-Villiger monooxygenase, but no whole cells. These have been lysed chemically or mechanically and preferably removed, e.g. by centrifugation.

In the conversion, oxygen is reduced to water and the co-factor NADPH is oxidized to NADP⁺. For an economically feasible process, the co-factor needs to be recycled. This can be achieved via a second enzymatic reaction, in which glucose is converted to gluconic acid by a glucose dehydrogenase (GDH). In this reaction NADP⁺ is reduced back to NADPH, which is then available again for the BVMO.

In a preferred embodiment of the conversion with a cell-free enzyme formulation described above, glucose and glucose dehydrogenase are present during the conversion.

In a further preferred embodiment of the method described in any of the embodiments above, the conversion is performed with a cell-free enzyme formulation at a temperature between 2° and 40° C., preferably between 25 and 30° C. and most preferably at 28° C.

In another preferred embodiment of the method described in any of the embodiments above, the conversion is performed with a cell-free enzyme formulation at a pH between 7.0 and 9.0, preferably at a pH between 7.5 and 8.5 and most preferably at a pH value of 8.0.

In another preferred embodiment of the method described in any of the embodiments above, the conversion is performed with a cell-free enzyme formulation at a substrate concentration between 1 and 20% (w/w), preferably between 5 and 15% (w/w), more preferably between 7 and 13% (w/w) and most preferably at a substrate concentration of 10% (w/w).

The above mentioned conditions have been found to provide particularly good results for the cell-free conversion.

Besides the option of using cell-free enzyme formulations comprising the Baeyer-Villiger monooxygenase, the use of whole cells expressing the Baeyer-Villiger monooxygenase for the biotransformation has some advantages. In particular, the in vivo regeneration of the expensive co-factor represents a significant advantage.

E. coli cells, which have been transformed with the BVMO gene as defined above under the control of a lac operator can be used for the whole cell transformation. Expression of the BVMO can thus be induced by adding isopropyl-beta-D-thiogalactopyranoside (IPTG). In principle, growing or resting cells can be used. Resting cells can be generated by transfer in phosphate buffer. In the context of the present invention, experiments were conducted in an auto-induction medium with growing cells and in phosphate buffer with resting cells. The auto-induction medium comprises—besides glucose as primary energy source—also glycerol and lactose. As long as glucose is present in the medium, the BVMO gene is repressed and maximum growth is observed at 37° C. When the glucose is used up, auto-induction of the BVMO gene by lactose occurs at 30° C. The reduced expression temperature avoids the formation of inclusion bodies, i.e. aggregates of denatured enzyme, which are not able to perform efficient conversion. Optimal temperatures for the expression are between 25 and 30° C. This setup is much easier to handle than a manual induction with IPTG. The amount of lactose is chosen so that a high expression is achieved. This process is automatic and the concentration of biomass until the induction can be adjusted by the initial glucose concentration. During the cultivation, ampicillin is added as selection marker so that the BVMO carrying plasmid is replicated by E. coli. After 15 to 20 hours of growth in the auto-induction medium and 2 to 3 hours after induction, the highest enzyme activities are observed, while the optical density still increases indicating an increase in biomass.

In another embodiment of the method described in any of the embodiments above, the conversion is performed with whole cells expressing the Baeyer-Villiger monooxygenase.

In a preferred embodiment, the whole cells are E. coli cells, which were transformed with agene encoding a Baeyer-Villiger monooxygenase as defined above.

Further preferred is an embodiment of the method described in any of the embodiments above, wherein the conversion is performed with whole cells expressing the Baeyer-Villiger monooxygenase and wherein the conversion is performed in the presence of EDTA, preferably in a concentration of 1 to 10 mM, more preferably 3 to 7 mM.

In a further preferred embodiment of the conversion performed with whole cells expressing the Baeyer-Villiger monooxygenase according to any of the embodiments described above, the mass ratio of dry biomass to EDTA is in a range from 1 to 3, preferably 1.5 to 2.5.

EDTA binds magnesium ions, which stabilize the outer membrane of the cell. Therefore, the addition of EDTA in the whole cell biotransformation significantly enhances the permeability of the cell membranes for the hydrophobic substrates and thus makes the conversion more efficient.

It was found out that the use of resting cells leads to a higher productivity compared to the use of growing cells when an energy source is added to the phosphate buffer, so that sufficient NADPH can be generated. The resting cells can only convert a limited amount of ketone in phosphate buffer. The addition of glucose leads to more conversion but a high glucose concentration results in inhibition of the reaction. Presumably, the addition of glucose leads to an increased generation of NADPH from the pentose phosphate pathway. Besides the pentose phosphate pathway, which metabolizes about 20% of glucose in E. coli according to literature, the glucose metabolism proceeds mainly via glycolysis, which does not provide any NADPH.

It is known that E. coli ferments aerobically at high glucose concentrations (Crabtree effect) and acetate, formate and other fermentation products are formed. The generation of these acidic substances leads to a quick acidification of the medium, which can inhibit the growth of the cells. This problem can be avoided by using gluconate as energy source. This allows to keep the pH more stable.

In a further preferred embodiment, the cells used in the whole cell conversion are resting cells and the conversion is performed in the presence of gluconate as energy source. Preferably, in this embodiment, the conversion is performed in a phosphate buffer.

Another factor, which plays a role in the productivity of the whole cell transformation is the biomass. The concentration of dry biomass during the conversion should be between 1.5 and 3.5 g/L, preferably between 2 and 3 g/L to provide a good productivity. Higher concentrations of biomass result in substrate limitation. However, if a fed-batch setup is used, which avoids critical substrate concentrations, a much higher cell concentration is possible and the dry biomass may be up to 70 g/L In a preferred embodiment of the method described in any of the embodiments above, wherein the conversion is performed with whole cells expressing the Baeyer-Villiger monooxygenase, the conversion is performed in a fed-batch set up and the dry bio mass is in a range from 20 to 70 g/L, preferably 30 to 60 g/L.

In the fed-batch setup, the obtained lactone concentrations are between 13 to 16 g/L at a reaction time of 10-12 h and the space-time yields are between 1.3 to 1.4 g/(L*h) The lactones are present within and outside of the cells and can be extracted with methyl tert-butyl ether (MTBE).

The conversion of lager volumes or with high cell density also benefits from additional oxygen supply. It is therefore preferred in this case that the conversion with whole cells expressing the Baeyer-Villiger monooxygenase is supplemented with oxygen.

In a further preferred embodiment of the method described in any of the embodiments above, wherein the conversion is performed with whole cells expressing the Baeyer-Villiger monooxygenase, the conversion is performed at a temperature between 2° and 30° C., preferably 22 to 28° C.

Since the ketones are not very soluble in water, the addition of a surfactant such as Tween 80 can increase the bioavailability in the whole cell biotransformation. In a further preferred embodiment of the whole cell conversion according to any of the embodiments described above, a surfactant or emulsifier is present during the conversion.

Furthermore, an excess of enzyme should be used in the fed-batch setup to obtain good productivity. In a further preferred embodiment of the method using whole cells, the enzyme is therefore used in excess.

The above mentioned conditions have been found to provide particularly good results for the whole cell conversion.

The present invention also relates to the use of a Baeyer-Villiger monooxygenase, preferably a Baeyer-Villiger monooxygenase as defined above, for the conversion of one or a mixture of two or more compound(s) of formula I to one or a mixture of two or more compound(s) of formula II and/or III:

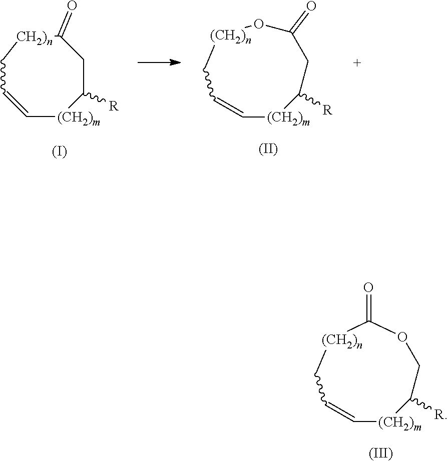

wherein the compound(s) of formula (I) is/are selected from the group consisting of the compounds (1) to (9):

| compound | n | m | R |
|---|---|---|---|
| (1) | 6 | 4 | H |
| (2) | 9 | 1 | H |
| (3) | 7 | 3 | H |
| (4) | 8 | 2 | H |
| (5) | 10 | 0 | H |
| (6) | 9 | 0 | H |
| (7) | 9 | 0 | CH₃ |
| (8) | 8 | 1 | CH₃ |
| (9) | 7 | 1 | CH₃ |

with the proviso that, in case only compound (1) is converted, the conversion is performed with whole cells expressing the Baeyer-Villiger monooxygenase.

Preferably, the Baeyer-Villiger monooxygenase is used in a method according to any of the embodiments described above. In particular, the Baeyer-Villiger monooxygenase used is preferably defined as in the context of the methods described above and the use may be by application of a cell-free enzyme formulation or whole cells expressing the Baeyer-Villiger monooxygenase The details described above in the context of the method in all its embodiments apply accordingly.

The invention also relates to a compound or mixture of two or more compounds selected from the group consisting of compounds (10) to (18)

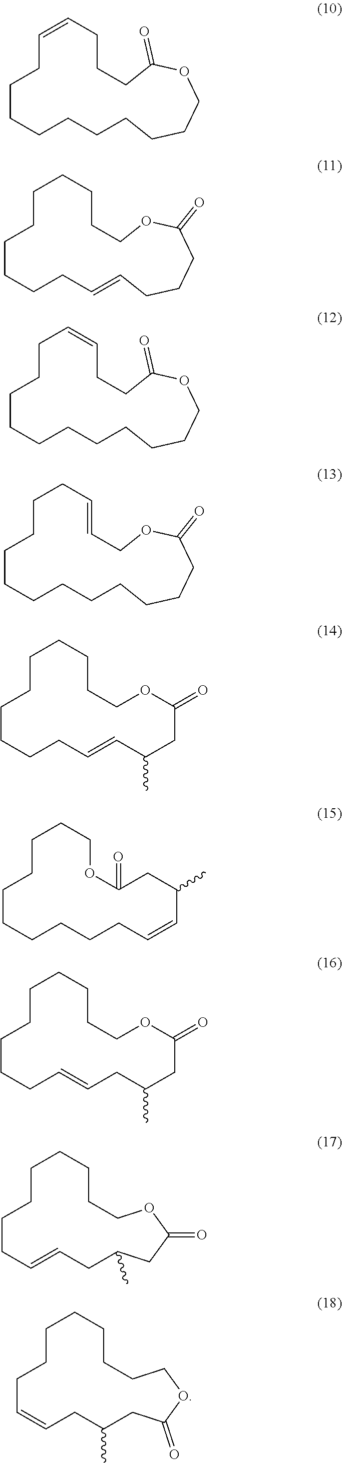

The above mentioned macrocyclic lactones are efficiently accessible with the method according to the present invention and they have not previously been described in the prior art.

The invention also relates to the use of a compound or mixture of compounds selected from the group consisting of compounds (10) to (18) as a fragrance.

Furthermore, the present invention relates to the use of a compound or mixture of two or more compounds selected from the group consisting of compounds (19) to (22) as a fragrance

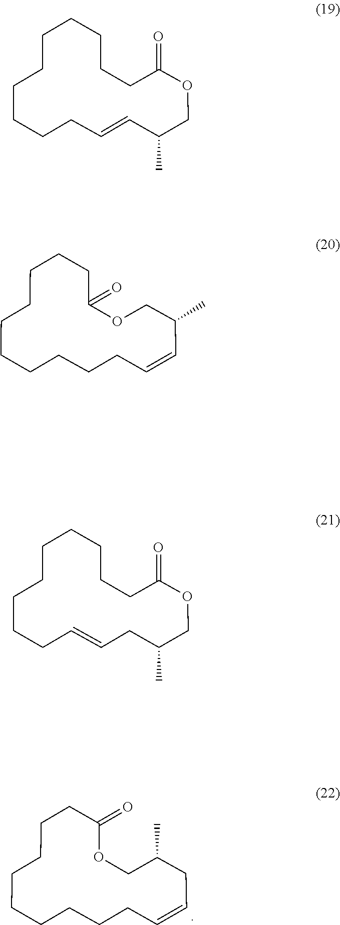 [SMILES: C[C@@H]1/C=C/CCCCCCCCCCC(=O)OC1 ; C[C@@H]1/C=C\CCCCCCCCCCC(=O)OC1 ; C[C@@H]1C/C=C/CCCCCCCCCC(=O)OC1 ; C[C@@H]1C/C=C\CCCCCCCCCC(=O)OC1]

Also the compounds (19) to (22) are readily accessible by the methods described above and can be used as fragrances.

Compounds (10) to (22) provide a musk fragrance.

### SHORT DESCRIPTION OF THE FIGURES

FIG. 1 shows the BVMO, NADPH and Globanone® dependent absorbance decrease in spectro-photometric assays at 340 nm.

FIG. 2 shows the GC chromatogram for the Aurelione® starting material used in examples 2 and 3

FIG. 3 shows the GC-MS chromatogram for Aurelione® starting material used in examples 2 and 3

FIG. 4 shows the mass spectrum of the first peak from the GC-MS chromatogram in FIG. 3.

FIG. 5 shows the GC-FID chromatogram reaction mixture after 20 h (see example 2).

FIG. 6 shows the GC-FID overlay of Aurelione® (grey) and the reaction mixture after 20 h (black) (see example 2).

The invention is further characterized by the following examples:

### EXAMPLE 1: ENZYME PRODUCTION

The genes for the two Baeyer-Villiger Monooxygenases (BVMOs) cyclopentadecanone monooxygenase from a Pseudomonas species (CPDMO, Iwaki, H. et al., Appl. Environ. Microbiol. (2006), 72 (4): 2707-2720) and cyclododecanone monooxygenase from a Rhodococcus species (CDMO, Kostichka, K. et al., J. Bacteriol. (2001), 183 (21): 6478-6486) were cloned using standard molecular biology tools. For recombinant enzyme production in Escherichia coli an L-arabinose inducible vector derived from a pBAD plasmid carrying a kanamycin/neomycin resistance gene was used in an E. coli K-12 strain, in which the Larabinose catabolism is disrupted. Glycerol stocks were used to inoculate 5 ml overnight cultures (LB, 100 μg/ml neomycin). Overnight cultures were shaken at 28° C. 0.5 ml of the overnight cultures was used to inoculate 50 ml LB medium (100 μg/ml neomycin) in 0.5 I shake flasks with foam top. Cultures were grown on an orbital shaker at 28° C. at 110 rpm. Induction by addition of L-arabinose (final concentration 0.02% (w/v) was done ca. 4 h after inoculation of the main culture. After induction, cultures were further cultivated at 28° C. In total, the main culture was grown 24 h at 110 rpm. Cells were harvested by centrifugation at 5000 rpm and 4° C. for 20 minutes. Cell pellets were weighed and frozen at −20° C. E. coli cell lysates with expressed CPDMO and CDMO, respectively, were prepared by addition of twice the weight of lysis buffer to the cell pellets. The lysis buffer was prepared by mixing 25 ml 100 mM MOPS buffer pH 7.5, 5 mg DNAse I, 100 mg lysozyme, 62 mg MgSO4 heptahydrate, 77 mg dithiothreitol (DTT) and 25 ml H2O. After resuspension, the cells were shaken 1 hour at room temperature and subsequently centrifuged at 17,000×g and 4° C. for 30 minutes. The cleared lysate supernatant was used for further studies.

Protein concentrations of the lysates were determined to calculate specific activities (Protein determination by Bradford). The concentrations of total protein in the cell-free extracts (CFEs) were determined using a modified protein-dye binding method as described by Bradford in Anal. Biochem. 72: 248-254 (1976).

Of each sample 50 μl in an appropriate dilution was incubated with 950 μl reagent (100 mg Brilliant Blue G250 dissolved in 46 ml ethanol and 100 ml 85% ortho-phosphoric acid, filled up to 1 I with Milli-Q water) for at least five minutes at room temperature. The absorption of each sample at a wavelength of 595 nm was measured in a PerkinElmer Lambda 35 UV/VIS spectrophotometer. Using a calibration line determined with solutions containing known concentrations of bovine serum albumin (BSA, ranging from 0.0125 mg/ml to 0.20 mg/ml) the protein concentration in the samples was calculated.

### Spectro-Photometric Activity Assay:

To determine if the two BVMOs were active on Globanone® a spectro-photometric activity assay was applied. The assay makes use of the strong absorbance decrease at 340 nm upon consumption of the NADPH cofactor. Compared to the Iwaki publication the substrate concentrations were lowered to 0.25 mM. The activity assay was conducted in a PerkinElmer Lambda 35 spectro-photometer thermostated to 30° C. at a wavelength at 340 nm in 1 ml cuvettes. The assays on Globanone® contained the following components: 47 mM Bis-Tris-propane buffer pH 9.0, 0.3 mM NADPH, 50 μl cell-free lysate and 0.25 mM Globanone®. All components except the substrate were pipetted together and equilibrated to 30° C. for 5 min. The assay was started by addition of 2.5 μl of substrate stock solution in n-propanol to the cuvette. The decrease of absorbance was followed for 3 minutes before substrate addition to detect potential, unspecific NADPH oxidation. After starting the reaction by substrate addition the absorbance was followed for additional 4-5 minutes and the BVMO activity was measured from the linear range of absorbance decrease at 340 nm (ΔAbs/min). Via the molar extinction coefficient of NADPH of ε=6.22 cm2/μmol the substrate concentration decrease and thus the volumetric activity in pmol substrate converted per minute and ml cell-free extract is calculated according to Lambert-Beer using the following formula:

$$Volumetric activity ( in U / ml or μmol / {min}^{*} ml ) = ( Δ Abs / {min}^{*} {V}^{*} D ) / ( {ε}^{*} {d}^{*} v )$$

with V=reaction volume (1000 μl), v=sample volume (50 μl), D=dilution factor of the enzyme sample (the factor for a 1/100 dilution is 100), E=6.22 cm2/pmol, d=path-length of the cuvette (1 cm)

The specific activity (in U or μmol/min per mg total cell-free lysate protein) was calculated by dividing the volumetric activity of a sample by its total protein concentration:

$$Specific activity ( U / mg ) = volumetric activity ( U / ml ) / protein concentration ( mg / ml )$$

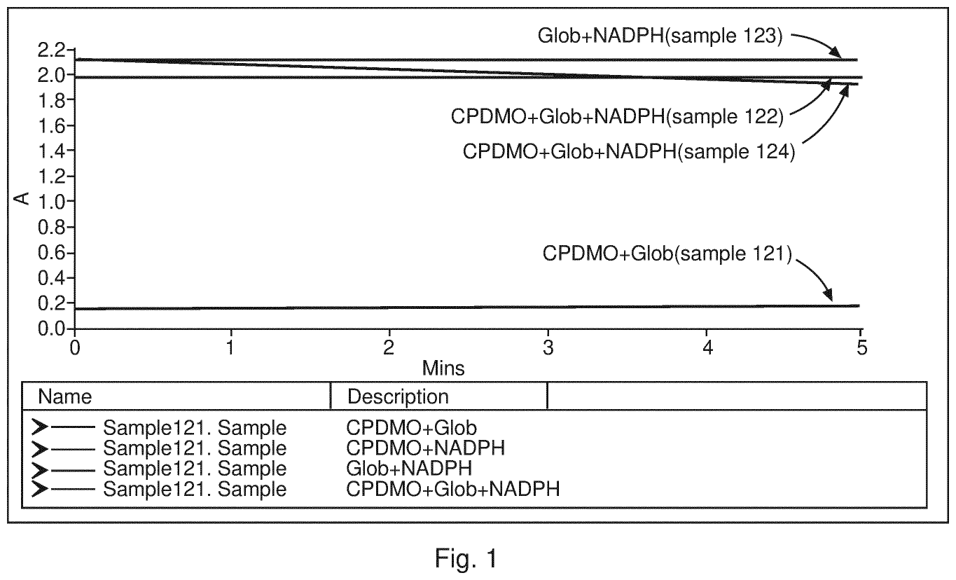

For the cell-free lysate containing CDMO a protein concentration of 18.6 mg/ml and a volumetric activity of 3.71 U/ml were determined resulting in a specific activity of 0.20 U/mg. For CPDMO a protein concentration of 9.9 mg/ml and a volumetric activity of 0.04 U/ml were determined resulting in a specific activity of 0.004 U/mg. In control reactions always one of the reactants was omitted to test if the absorbance decrease is strictly dependent on the presence of (i) BVMO enzyme, (ii) Globanone® substrate and (iii) the NADPH redox cofactor. FIG. 1 clearly demonstrates that only if all reaction components are present in the assay gives a significant absorbance decrease, while the other assays with only two components give straight lines with sometimes minimal baseline fluctuation. As the assay detects the consumption of the cofactor, but not unequivocally the formation of the target product, an additional type of assay with GC-FID and GC-MS detection of the target product was performed.

### EXAMPLE 2: ANALYTICAL SCALE BVMO OXIDATION OF AURELIONE® STARTING MATERIAL

The substrate Aurelione® starting material was tested in the BVMO enzyme catalyzed oxidation. In addition to the E/Z-isomers of Globanone®, the Aurelione® starting material predominantly contains a Globanone® regio-isomer, having the double bond in the 7-postion instead of the 8-position like Globanone®, next to other Globanone® regio-isomers. As first step a GC analysis method was established to analyze the Aurelione® starting material:
Initial temperature: 150° C.
Hold: 0 min
Rate 1: 4° C./min
Temperature: 230° C.
Rate 2: 25° C./min
Final temperature: 300° C.
Hold: 2.2 min
Injector temperature: 280° C.
Detector temperature: 280° C.
Analysis time: 25 min.

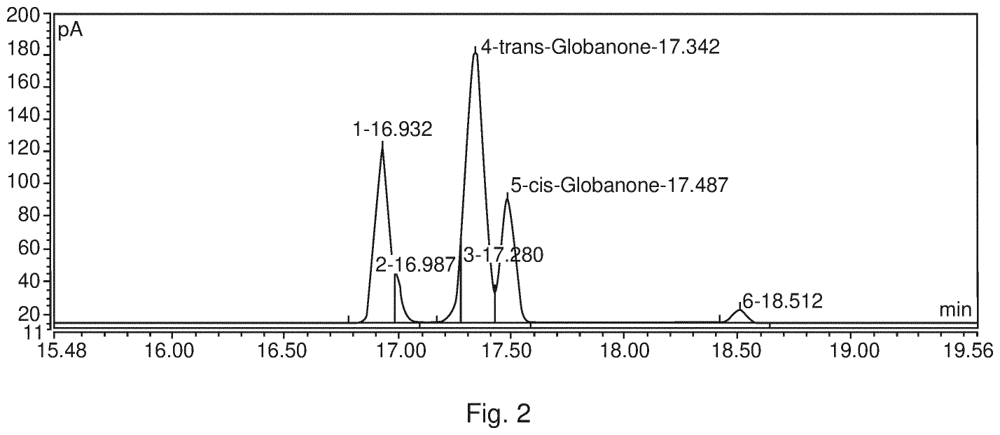

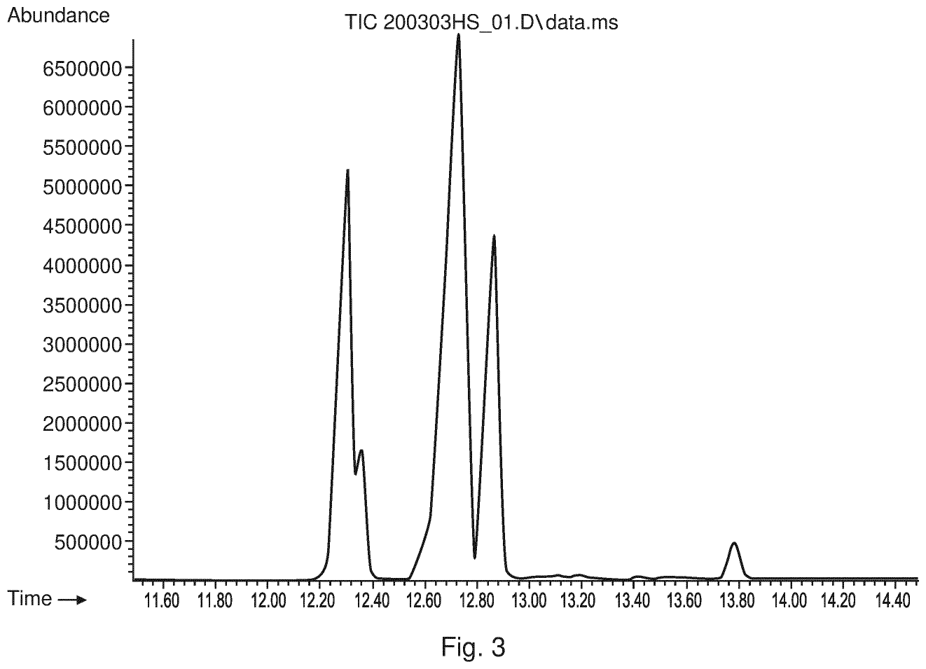

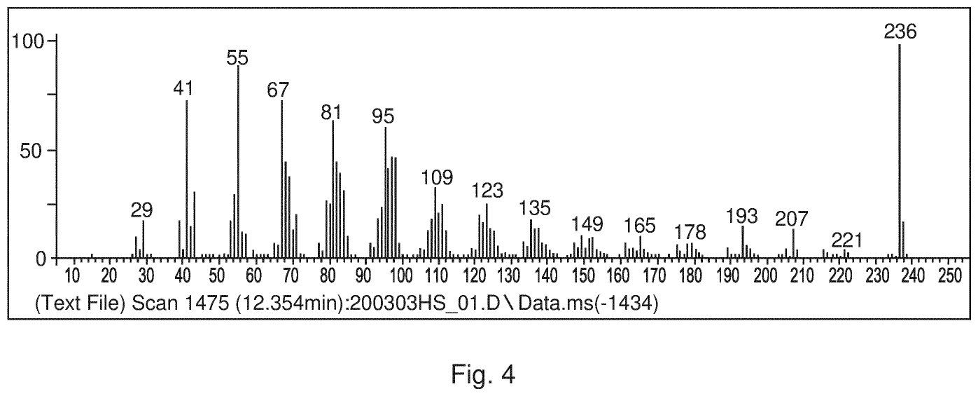

The GC-FID analysis of the starting material showed that the Aurelione® starting material consists of more than 4 isomers (FIG. 2). The presence and retention times of Globanone® (E and Z) were confirmed using available reference compounds. Co-elution of other isomers could not be excluded. Subjecting the same sample to GC-MS analysis showed a peak pattern comparable to that obtained with GC-FID (FIG. 3). For all peaks, an m/z of 236 and very similar fragmentation pattern were observed (FIG. 4).

A 40 ml experiment was carried out with an airflow set to ca. 6 ml/min. The pH was controlled and kept constant by titration with aqueous 5 M NaOH. Glucose (7.2 g), NADP+ (20 mg), and glucose dehydrogenase (GDH, 42 mg) were added to the reactor followed by the buffer solution (potassium phosphate (KPi) 50 mM, pH 8.0; 20 ml) and Tween-80 (2 g of 10% solution in KPi buffer). Subsequently, Aurelione® starting material was added (4.15 g; 10% (w/w)) followed by the E. coli cell-free extract (CFE) containing the BVMO enzyme (8 ml, 20% (v/v)). The reaction progress was monitored by GC-FID analysis of samples. The conversion was calculated based on area %. Full conversion was reached after ≤20 hours.

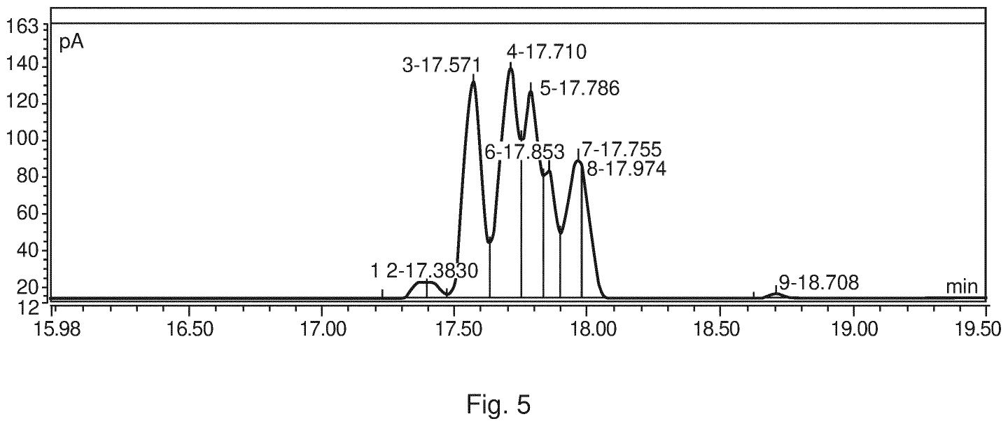

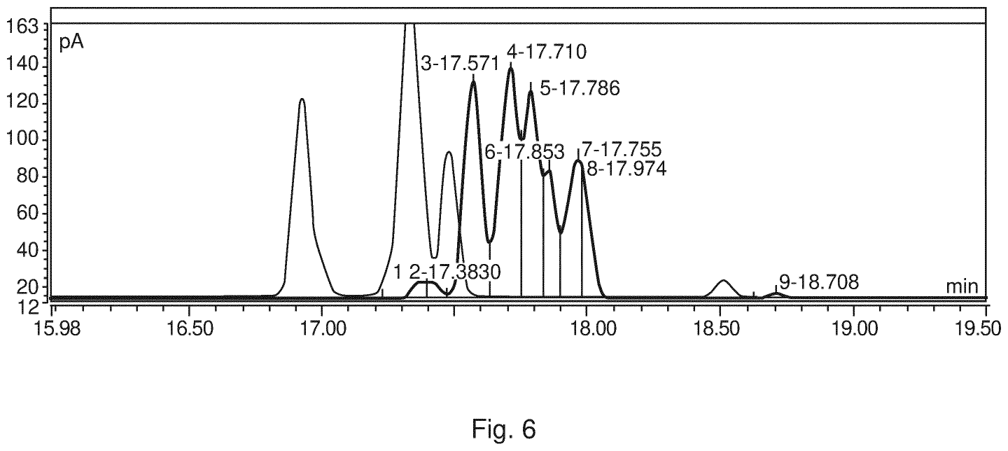

The GC-analysis (FIG. 5) showed that at least 9 different isomers were formed. There was co-elution of some of the ketone isomers of the starting material with some of the isomers of the lactones (FIG. 6).

GC-MS was used to confirm the production of isomers. The peak pattern of the GC-MS analysis was comparable to that of the GC-FID. No indications for the presence of unconverted ketones were observed.

### EXAMPLE 3: PREPARATIVE SCALE BVMO OXIDATION OF AURELIONE® STARTING MATERIAL

A 1 I scale BVMO oxidation reaction of Aurelione® starting material was performed. Glucose (120 g; 2 equivalents), NADP+ (0.4 g), and GDH (0.4 g) were added to the reactor, followed by the buffer solution (KPi 50 mM, pH 8.0; 500 ml) and Tween-80 (40 g of 10% solution in buffer). Subsequently, Aurelione® starting material was added (75 g; 8% (w/w)), followed by the BVMO CFE (200 ml, 22% (v/v)). The airflow was initially set at 75 ml/min for the first 2 h of the reaction and then reduced to 35 ml/min and further to 25 mL/min after 6 h to run the reaction overnight. The stirrer speed was increased from 350 rpm to 500 rpm. The pH was controlled at 8.0-8.3 and kept constant by titration with aqueous 5 M NaOH. The reaction progress was monitored by GC-FID analysis. After 22 h quantitative conversion of the Aurelione® starting material was observed.

The reactor was discharged and rinsed with heptane (1 I). The reactor content and the heptane rinse were combined and transferred to a 4 I reactor. The mixture was heated to 70° C. under gentle stirring. After three hours, the mixture was allowed to settle (overnight), which resulted in 3 phases; a clear yellow water layer, an emulsion and a clear slightly yellow organic layer. The water layer (600 ml) was separated and the organic layer was decanted. The remaining mixture was filtered over a pre-coated filter, using filter-aid (Dicalite 4208; 50 g) and sodium sulfate (25 g). The filter-cake was washed with heptane (500 ml). The biphasic filtrate and wash liquid were combined. After phase separation, the organic phase was combined with the original heptane phase that was decanted. The combined phases were filtered over a paper filter and concentrated to 75 g light yellow oil resulting in a yield on Aurelione® starting material of 93%.

### EXAMPLE 4: REACTION IN 30 L FERMENTER ON 15 L SCALE

The conversion of 1 kg Aurelione® starting material was performed in a 30 L fermenter, designed to efficiently supply air.

A buffer solution (KPi 50 mM, pH=8; 8 L) was added to the reactor and the stirrer was started. Subsequently, glucose (1600 g; 2eq), NADP (7.5 g), GDH (7.5 g) and tween-80 (750 g of 10% solution in buffer) were added. When the glucose was dissolved, Aurelione® starting material was added (1010 g as a liquid after melting at 37° C.; 7 w %). Finally, the BVMO (3000 ml, cell-free extract from fermentation broth; 22 v %) and antifoam agent (5 g) were added. The airflow was initially set at 3 L/min and the stirrer speed at 200 rpm. The pH was controlled at 8.0-8.3 by titration with aqueous 5 M NaOH. As before, reaction progress was monitored by GC-analysis of samples.

After 2 h the stirrer speed was increased to 250 rpm, which increased the oxygen uptake. The airflow was gradually decreased after 6 h to 1 L/min after 8 h. The reaction proceeded faster than the 1 L scale experiment because of the higher airflow that could be applied. After 8 hours, 95% conversion was reached. Additional glucose was added (800 g) and stirred was continued overnight, which resulted in full conversion. The NaOH titration continued, as expected, even after full conversion was reached.

The reaction mixture was split into three portions, each approximately 6 L. The portions were separately worked up in a 10 L reactor. Heptane (3 L) was added to each portion and the mixture was heated up to 70° C. while gently stirring. After 2 hours the stirring was stopped and the layers were allowed to settle for 1 hour. Three layers were obtained. The top layer (heptane extract) was decanted as much as possible by means of vacuum. The bottom layer (water waste) was separated as much as possible. The remainder of the reactor content was transferred to an Erlenmeyer flask of 4 L. Dicalite 4208 (150 g, filter-aid), and sodium sulfate (100 g) were added. The mixture was stirred for 15 minutes. A glass filter (P3, d=20 cm) was precoated with Dicalite 4208 (1 cm). The mixture was filtered and the filter-cake was washed with 3×300 mL heptane. The filtrate and wash liquids were combined and allowed to settle. After phase separation, the organic phases was combined with the original heptane top phase. The combined organic phases were filtered over a paper filter and subsequently concentrated to a somewhat turbid yellowish oil; ˜350 g.

A second filtration was performed to remove the turbidity from the isolated product. The combined isolated oils 1055 g (containing 4% heptane) in total—were taken up in heptane (1 L). Dicalite 4208 (50 g) and sodium sulfate (25 g) were added. The mixture was stirred for 15 minutes and subsequently filtered over a precoated glass-filter (P3). The filter-cake was washed with heptane (2×200 mL). The combined filtrate and wash liquids were concentrated to 1040 g of yellowish clear oil (containing 2.7% heptane). This oil was combined with the 35 g of product from the second 1 L-scale experiment and further concentrated using an oil pump to remove the residual heptane. Finally 1052 g clear yellow oil was obtained. The overall yield amounts to 91%, corrected for assay (96%, vide infra) and the addition of 35 g oil from the 1 L experiment.

### EXAMPLE 5: WHOLE CELL BIOTRANSFORMATION

A plasmid encoding the BVMO gene from Pseudomonas spec HI-70 was transformed into an E. coli BL12 strain. The plasmid was codon optimized for the expression in E. coli and additionally carries as a promoter the Lac gene as well as an ampicillin resistance as selection marker.

The simplest whole cell biotransformation is the addition of substrate after induction of the BVMO in auto-induction medium with growing cells. In addition, EDTA was added after the induction. A 500 ml Erlenmeyer flask with 100 ml auto-induction medium was used at 28° C. The conversion was performed at a pH of 7 to 7.5 because E. coli does not show significant growth at pH 9. This pH value is under the optimum for BVMO. After a growing phase and induction of the BVMO, EDTA and Globanone® (compound (1)) were added. The ketone was added in ethanol.

A comparison shows that EDTA significantly increases the reaction speed and the yield in an auto-induction medium. The optimal EDTA concentration is at 5 mM. The mass-ratio dry biomass/EDTA is about 2. The initial addition of Globanone® leads to less conversion than the addition only after induction (13 h). Using 5 mM EDTA, there is 1.5 mmol lactone/g dry biomass at 48% conversion. This corresponds to about 0.38 g lactone/g dry biomass.

### EXAMPLE 6: HIGH DENSITY WHOLE CELL CONVERSION IN A FERMENTER

After important reactions conditions have been established in an Erlenmeyer flask, the high cell density fermentation was optimized on a 2 L scale and the bioconversion on a 4 L scale.

The process can be divided into two phases. The first phase comprises the growth of biomass to obtain sufficient amounts of catalyst for the biotransformation. The biotransformation is performed in an inexpensive phosphate buffer. Using a fed-batch setup, it is possible to reach high concentrations of biomass. A high cell density fermentation can lead to biomass of up to 70 g/L. After about 6 hours, the maximum growth rate μₘₐₓ is adjusted to a lower growth rate μₛₑₜ by glucose limitation to avoid the Crabtree effect (μₘₐₓ: 0.4 h⁻¹ and μₛₑₜ: 0.22 h⁻¹). The second phase comprises the optimization of the biotransformation on a 1 to 2 L scale. It was found that this can be optimally performed in phosphate buffer. To achieve a quick and complete conversion, an excess of enzyme has to be used. The larger the excess, the faster the reaction progresses. This way, kinetics determined by substrate limitation can be avoided. Ideally, during the biotransformation, oxygen should be supplied because the high biomass requires large amounts of oxygen. Very important is also the substrate concentration. A substrate feeding process was established, which allows to avoid any critical substrate concentration. Substrate concentrations >30 mM ketone inhibit the biotransformation. Higher temperatures lead to a faster reaction but also to a faster inactivation of the BVMO.

The optimization was performed with Velvione® (compound (2)) as substrate. It was established that an excess of enzyme has to be used to obtain a high conversion rate and that the substrate concentration should not be too high at any point since this may lead to inhibition of the conversion. Using the fed-batch process, it is possible to obtain up to 15 g/L lactone in 10 to 12 hours.

1. A method for converting one or a mixture of two or more compound(s) of formula I to one or a mixture of two or more compound(s) of formula II and/or III: 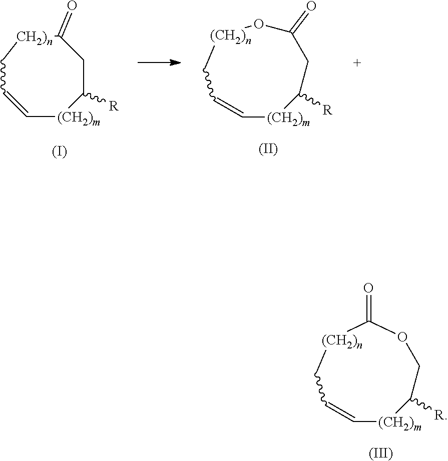
wherein the compound(s) of formula (I) is/are selected from the group consisting of the compounds (1) to (9):

| compound | n | m | R |
|---|---|---|---|
| (1) | 6 | 4 | H |
| (2) | 9 | 1 | H |
| (3) | 7 | 3 | H |
| (4) | 8 | 2 | H |
| (5) | 10 | 0 | H |
| (6) | 9 | 0 | H |
| (7) | 9 | 0 | CH₃ |
| (8) | 8 | 1 | CH₃ |
| (9) | 7 | 1 | CH₃ |

wherein the conversion is catalyzed by a Baeyer-Villiger monooxygenase, with the proviso that, in case only compound (1) is converted, the conversion is performed with whole cells expressing the Baeyer-Villiger monooxygenase.

2. The method according to claim 1, wherein a mixture of the compounds (1) and (3), optionally further comprising compound(s) (4) and/or (5), is converted.

3. The method according to claim 1, wherein the Baeyer-Villiger monooxygenase is a cyclopentadecanone monooxygenase derived from a Pseudomonas species or a cyclododecanone monooxygenase derived from a Rhodococcus species.

4. The method according to claim 1, wherein the Baeyer-Villiger monooxygenase has an amino acid sequence selected from the sequences of SEQ ID NOs: 3 and 6, or an amino acid sequence having a sequence identity of at least 80% to any of the sequences of SEQ ID NOs: 3 and 6.

5. The method according to claim 1, wherein the Baeyer-Villiger monooxygenase is encoded by a nucleic acid sequence selected from the sequences of SEQ ID NOs: 1, 2, 4 and 5, or a nucleic acid sequence having a sequence identity of at least 80% to any of the sequences of SEQ ID NOs: 1, 2, 4 and 5.

6. The method according to claim 1, wherein the conversion is performed with a cell-free enzyme formulation comprising the Baeyer-Villiger monooxygenase.

7. The method according to claim 6, wherein the conversion is performed at a temperature between 2° and 40° C., and/or the conversion is performed at a pH between 7.0 and 9.0, and/or the conversion is performed at a substrate concentration between 1 and 20% (w/w).

8. The method according to claim 1, wherein the conversion is performed with whole cells expressing the Baeyer-Villiger monooxygenase.

9. The method according to claim 8, wherein the whole cells are E. coli cells, which were transformed with a gene encoding a Baeyer-Villiger monooxygenase.

10. The method according to claim 8, wherein the conversion is performed in the presence of EDTA.

11. The method according to claim 8, wherein the cells are resting cells and the conversion is performed in the presence of gluconate as energy source.

12. (canceled)

13. A compound or a mixture of two or more compounds selected from the group consisting of compounds (10) to (18)

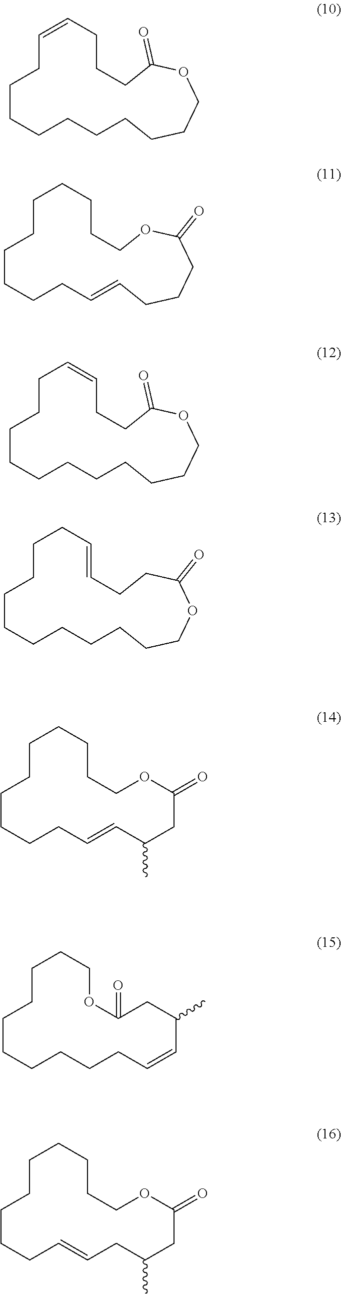

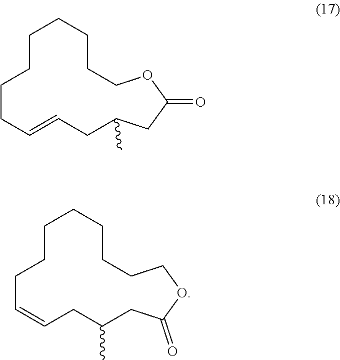

14. (canceled)

15. (canceled)

16. The method according to claim 6, wherein the conversion is performed at a temperature between 25 and 30° C.

17. The method according to claim 6, wherein the conversion is performed at a pH between 7.5 and 8.5.

18. The method according to claim 6, wherein the conversion is performed at a substrate concentration between 5 and 15% (w/w).

19. The method according to claim 6, wherein the conversion is performed at a substrate concentration between 7 and 13% (w/w).

20. The method according to claim 8, wherein the conversion is performed in the presence of 1 to 10 mM EDTA.

21. The method according to claim 8, wherein the conversion is performed in the presence of 3 to 7 mM EDTA.
